(19) United States
(12) Patent Application Publication
(10) Pub. No.: US 2005/0276781 A1
(43) Pub. Date: Dec. 15, 2005

(54) Molecularly imprinted phosphate binders for therapeutic use

(76) Inventors: Edward Allan Ross, Gainesville, FL (US); Christopher D. Batich, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/148,929

(22) Filed: Jun. 9, 2005

Related U.S. Application Data
(60) Provisional application No. 60/578,693, filed on Jun. 9, 2004.

(51) Int. Cl. A61K 31/74

(52) U.S. Cl. 424/78.08; 524/90

(57) ABSTRACT

Methods for synthesizing molecularly imprinted polymers (MIP) having an affinity for dietary phosphates, resulting polymers, pharmaceutical compositions and modes of administration are disclosed. The MIP compounds are useful for binding excess dietary phosphates in a patient in need thereof.

### CROSS-REFERENCE TO RELATED APPLICATION

The present application claims benefit of U.S. Provisional Application Ser. No. 60/578,693, filed Jun. 9, 2004, which is hereby incorporated by reference herein in its entirety, including any figures, tables, nucleic acid sequences, amino acid sequences, and drawings.

### BACKGROUND OF INVENTION

The field of the present invention relates to methods of synthesizing self-assembled polymers molecularly imprinted to targeted molecules, for example, dietary phosphates and the polymers thereof. The field of the present invention also relates to methods for administering the polymers to patients suffering from renal insufficiency. In one embodiment of the method, the polymers are administered to treat hyperphosphatemia.

Loss of renal function and excretion inevitably leads to the accumulation of a number of compounds absorbed from the diet. Retention of dietary phosphate leads to high blood levels of serum phosphorus resulting in widespread ramifications across many organ systems. For example, hyperphosphatemia causes imbalances in calcium and parathyroid hormone homeostasis and leads to progressive vascular and bone disease, which in turn contribute to the profound morbidity and mortality from which these patients suffer.

The adverse clinical consequences of retained phosphate are indeed so great that there are burgeoning pharmaceutical industries addressing this problem. While much effort has been focused on preventing or remediating damage cause by hyperphosphatemia, the preferred approach is to avoid high blood levels of that chemical by decreasing gut absorption. Unfortunately, until recently, all oral phosphate binders have had serious side-effects when used at high doses for prolonged periods of time.

Currently, a synthetic polymer, sevelamer hydrochloride (RENAGEL), has become the most popular binder because it is effective, nonabsorbable and without known adverse effects. Unfortunately, it is without a comparable competitor, is expensive, and requires multiple tablets because of the limited potency of the native compound.

There is a remarkable paucity of safe marketed products to address this great need. There was a time when it was believed that the relatively inexpensive aluminum-based and calcium-based binders were safe, even in high doses; that has proven to be incorrect. The aluminum products are indeed toxic and relatively contraindicated now. Calcium carbonate (over the counter) and calcium acetate (prescription only) are efficacious, but their doses are limited by recent guidelines capping the total daily oral calcium intake in this patient group. Similarly, magnesium based compounds are effective in the laboratory, but are impractical for patients because the total permitted daily dosage is limited by toxicity. The only nonabsorbable effective product is the prescription-only polymer sevelamer HCl, which is thereby enjoying incredible market share despite its high cost. Its affinity to phosphate is not ideal, however, and this leads to high dosing requirements. Dialysis patients often take 4-6 tablets with each meal, every day. Each tablet costs between $1.05 and $1.10, making it an extraordinarily profitable medication.

### BRIEF SUMMARY

One aspect of the present invention provides methods for synthesizing molecularly imprinted polymers (MIP) compounds having an affinity for dietary phosphates, the MIP compounds themselves, and uses thereof. Preferably, the MIP compounds are macroporous and have specific binding capacity for dietary phosphates. Macroporosity may be achieved, for example, by physically spacing the active binding sites by incorporating at least one pore forming diluent into the monomer mixture (e.g., a non-polymerizable solvent such as hexane or isopropanol). This material can be extracted or volatilized to leave pores in the polymer, thereby increasing the surface area available to interact with aqueous phosphate solutions.

Yet another aspect of the present invention provides MIP compounds synthesized in accordance with the methods of the present invention. The MIP compounds of the present invention comprise one or more polymerized monomers having a plurality of active binding sites for phosphates, and cross-linking agents that structurally support the polymer.

A different aspect of the present invention provides methods for reducing dietary phosphate absorption by administering a MIP compound of the invention to a patient in need thereof, in an amount sufficient to bind excess dietary phosphates in the patient's gastrointestinal (GI) tract. Preferably, the patient ingests a MIP compound of the invention immediately before eating, during eating, or immediately after eating. Preferably, the MIP compounds are dispensed in a particulate form and packaged in a pharmaceutical composition, such as a capsule, tablet or sprinkled powder wherein the patient ingests the MIP compound (e.g., while eating). Thus, the method of the present invention is useful for treating and/or reducing the severity of hyperphosphatemia (abnormally high serum phosphate levels).

### DETAILED DISCLOSURE

The present invention provides methods for synthesizing molecularly imprinting polymers (MIP) compounds useful for binding dietary phosphate, MIP compounds, and pharmaceutical compositions containing such compounds. The present invention also provides methods for reducing dietary phosphate absorption within a patient having elevated serum phosphate levels, or who is at risk thereof, by administering an MIP compound of the invention to the patient, in an amount sufficient to bind excess dietary phosphates in the patient's gastrointestinal (GI) tract. The methods of the present invention are useful for treating and/or reducing the severity of symptoms associated with hyperphosphatemia of various etiologies. The methods of the present invention are of therapeutic benefit to patients suffering, for example, from a disease associated with disorders of phosphate metabolism or a disease mediated by impaired phosphate-transport function.

One aspect of the present invention is directed to methods for the synthesis of MIP compounds having an affinity for binding phosphate or phosphate-containing molecules. The methods of the present invention comprise contacting the targeted imprint molecule (also referred to herein as the molecular template or print molecule) with a mixture of at least one monomer, at least one diluent, and at least one cross-linker (thereby forming a contacting mixture); polymerizing the contacting mixture with sufficient porosity to adequately expose the binding sites to the target molecule during administration; and removing the targeted imprint molecule and diluents. The targeted imprint molecule is phosphate, or a phosphate-containing molecule, such as potassium dihydrogen phosphate (KH₂PO₄), which is provided to allow imprint associations on the one or more monomers of the mixture. In a specific embodiment, the methods further comprise incorporating an inert physical spacer into the mixture that is not bound to the polymer but is dispersed among the polymer particles.

Monomers useful for the methods of the present invention preferably possess natural affinities to bind to different aspects of a phosphate-containing three-dimensional molecule. These monomers include, without limitation, allylamine, methylmethacrylate; hydroxyethylmethacrylate; N,N-di-ethylamino ethyl methacrylate; acrylic acid; alkyl methacrylate; alkylacrylates; arylacrylates; acrylamide; methacrylamide; N-methylacrylamide; N-methylmethacrylamide; styrene; para-hydroxy-styrene; para-amino-styrene; vinylpyridine; para-vinyl benzoic acid; 2-vinyl-2-hydroxypyridine; 3-vinyl-2-hydroxypyridine; 4-vinyl-2-hydroxypyridine; 4-vinylbenzamide; N-alkyl-(4-vinylbenzamide); N,N-dialkyl-(4-vinylbenzamide); N,N′-diethyl(4-vinylphenyl)amidine; acrylonitriles; ethacrylamide; alkacrylamides; alkyl substituted alkyl acrylates in general where the alkyl group is an aliphatic or aromatic group; butadiene; caprolactone; ethylene; propylene; divinylbenzene; ethylene glycol; propylene glycol; dimethylsiloxane; lactide; glycolide; ornithine; vinyl acetate; vinyl alcohol; vinyl chloride; vinyl isobutyl ether; vinyl methyl ether; urethane; isocyanates; isothiocyanates; dimethyl aminoethyl acrylate methyl chloride; [2-(methacryloyloxy)ethyl]trimethylammonium chloride; vinylpyrrolidone; and combinations of any of the foregoing.

Preferably, the monomers are acrylates or aromatics. More preferably, the acrylates are selected from the group consisting of methylmethacrylate; hydroxyethylmethacrylate; N,N-di-ethylamino ethyl methacrylate; acrylic acid; and mixtures thereof. More preferably, aromatics are selected from the group consisting of styrene, para-hydroxy-styrene, para-amino-sytrene, vinylpyridine, para-vinyl benzoic acid, and mixtures thereof. In one embodiment, about 3 to about 9 parts by volume of monomer to cross-linker is utilized. The choice of monomer will depend partially upon the polymerization technique utilized in the polymerizing step. Preferably, the monomers are polymerized using addition polymerization. However, other methods of polymerization, such as condensation polymerization, may be utilized. Generally, relatively polar (active) monomers are preferred over less polar (particularly, non-polar) monomers.

In a specific embodiment, at least two monomers are utilized according to the methods of the present invention. In one embodiment, the monomers are comprised of one part polar (active) monomer and one part less polar (relatively inactive) monomer. In yet another embodiment, the monomers are comprised of one part active monomer and two parts less polar, relatively inactive monomers. In a preferred embodiment, the active monomer is [2-(methacryloyloxy)ethyl]trimethylammonium chloride (METAC), and the less polar (relatively inactive) monomers are hydroxyethyl methacrylate (HEMA) and methyl methacrylate (MMA). In another embodiment, the polar monomer is allylamine (or 2-propen-1 amine).

Diluents useful for the methods of the present invention are selected to physically separate the monomers as the monomers polymerize, thereby physically spacing the active binding sites. The diluents are removed during subsequent purifications, for example, extractions and volatilizations, of the polymer leaving pores in the polymer. Advantageously, the pores create more surface area and expose additional binding sites. Examples of suitable diluents include, without limitation, methanol, ethanol, t-butanol, octanol, hexane, and isopropanol. In a specific embodiment, one part by weight inert diluent is added to one part by weight active monomer. In another specific embodiment, one part by weight each of two different inert diluents are added to one part by weight active monomer.

Cross-linkers of the present invention include, without limitation, difunctional acrylates, trifunctional acrylates, tetrafunctional acrylates, difunctional methacrylates, trifunctional methacrylates, tetrafunctional methacrylates, divinyl benzene, alkylene glycol, polyalkylene glycol diacrylates, methacrylates, dialkyldiglycol dicarbonate, dialkyl maleate, dialkyl fumurate, dialkyl itaconate, vinyl esters, ethylene glycol dimethacrylate, ethylene glycol diacrylate, diethylene glycol diacrylate, triethylene glycol diacrylate, tetraethylene glycol diacrylate, vinyl acrylates, vinyl methacrylates, alkyl acrylates, divinylbenzene, diallyldiglycol dicarbonate, diallyl maleate, diallyl fumarate, diallyl itaconate, vinyl esters, divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or bismethacrylamide, including hexamethylene bisacrylamide or hexamethylene bismethacrylamide, di(alkene)tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1,1-dimethyl-2-isocyanaotoethyl acrylate, tetraethylene glycol diacrylate, tetraethylene glycol dimethacrylate, triethylene glycol dimethacrylate, hexanediol dimethacrylate, and hexanediol diacrylate. Preferably, the cross-linker utilized in the methods of the present invention is di-ethylene glycol diacrylate.

In a preferred embodiment, the weight percentage of cross-linker in the MIP compounds of the present invention ranges from about 3% to about 35%. More preferably, the weight percentage of cross-linking agent in the MIP compound is about 10% to about 20%. Other exemplified weight percentages of cross-liner in the MIP compounds are about 3 wt %, about 4 wt %, about 5 wt %, about 6 wt %, about 7 wt %, about 8 wt %, about 9 wt %, about 10 wt %, about 11 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, or about 35 wt %.

The contacting step preferably takes place at a temperature within the range of about 17° C. and about 77° C. More preferably, the contacting step takes place at a temperature within the range of about 27° C. and about 57° C. Most preferably, the contacting step takes place at 37° C. Contacting continues for a period of time sufficient for the targeted imprint molecule to form imprint associations on the monomers. During the contacting step, the monomers self-assemble in their ideal configuration, i.e., the lowest energy state, to bind to different aspects of the targeted imprint molecule. In one specific embodiment, the contacting mixture is agitated at room temperature (˜23° C.) for at least three hours. In another specific embodiment, the contacting mixture is agitated at about 37° C.

The polymerizing step is initiated using initiators such as those known to those skilled in the art including, without limitation, ultraviolet or thermal free radical initiators such as peroxides, azo compounds, or redox based compounds. Preferably, the initiator is selected from the group consisting of benzoyl peroxide, acetyl peroxide, lauryl peroxide, azobisisobutyronitrile, 2,2′-Azobis(2-methylpropionamidine)dihydrochloride, t-butyl peracetate, cumyl peroxide, t-butyl peroxide, t-butyl hydroperoxide, bis(isopropyl)peroxy-dicarbonate, benzoin methyl ether, 2,2′-azobis(2,4-dimethylvaleronitrile), tert-butyl peroctoate, phtalic peroxide, diethoxyacetophenon, tert-butyl peroxyypivalate, diethoxyacetophenone, 1-hydroxycyclohexyl phenyl ketone, 2,2-dimethoxy-2-phenyl-acetophenone, phenothiazine, diisopropylxanthogen disulfide, and a combination of any of the foregoing. More preferably, the initiator is azo bis(2-methyl proplon amidine)di-hydrochloride.

The polymerizing step locks the monomers into the lowest energy state. The resulting polymer is electrostatically imprinted to the phosphate and is in a solid state. Preferably, conditions of the polymerizing step mimic absorption conditions of the GI tract. In one embodiment, the phosphate-monomer mixture is allowed to rest in a 50° C. oil bath. In another embodiment, the phosphate-monomer mixture is heated to a temperature within the range of about 17° C. to about 57° C. More preferably, the mixture is heated to a range within about 27° C. to about 47° C. Most preferably, the mixture is heated to about 37° C. In yet another embodiment, the polymerization step takes about 72 hours.

The removing step carried out according to the methods of the present invention removes the phosphates, unlinked monomers, unreacted reagents and inert diluents from the polymer. In one specific embodiment, the solid polymer is finely ground. Preferably, the finely ground particle size is about 200 microns to about 250 microns. In another specific embodiment, the polymer is converted to a semi-dried gel using techniques known in the art. Regardless of the physical state of the polymer, the polymer undergoes several purification steps including washings and elutions.

In a specific embodiment, removing the phosphates and diluents comprises carrying out a plurality of washings and filtrations, and alternating alcohols and deionized water as the washing media. In one embodiment, the alcohol washing media is isopropanol.

To remove any excess liquid from the polymer and to remove any volatile diluent, the polymer is dried before eluting the phosphates. In one embodiment, a vacuum oven preheated to about 45° C. to about 55° C. dries and degasses the polymer. Preferably, the oven is preheated to about 50° C.

In another embodiment, the polymer is freeze-dried, thereby removing any volatile diluents.

The eluting step comprises a plurality of washes to ensure that the phosphates are entirely removed from the polymer. Suitable washing techniques are known in the art. The eluting washing media can be, for example, an alcohol, an acid, a base or water. Preferably, the media is alcohol, hydrochloric acid or sodium hydroxide. In one embodiment, the plurality of washes comprises contacting the MIP compounds with the washing media, centrifuging until MIP pellets are formed, and decanting the supernatant. Eluting of the phosphates from the MIP compounds continues until the phosphate remaining in the supernatant is negligible. In a preferred embodiment, the washing media alternates between an alcohol and a strong base. Preferably, the alcohol is selected from the group consisting of methanol, ethanol, isopropranol and t-butanol. Preferably, the strong base is sodium hydroxide. In another preferred embodiment, the washing media alternates between an alcohol and an acid. Preferably, the alcohol is selected from the group consisting of methanol, ethanol, isopropranol and t-butanol. Preferably, the acid is hydrochloric acid.

Although pH is not critical, the removing step may further comprise adjusting the pH of the MIP compounds to a desired pH. In a preferred embodiment, the pH is basic. The pH can be adjusted by adding an acid and/or a base until the preferred pH is met. Preferably, the acid is hydrochloric acid. Preferably, the base is sodium hydroxide.

The incorporating step carried out according to the methods of the present invention is directed to adding a physical spacer to the finely ground polymer. In one embodiment, the physical spacer is added before eluting the phosphate from the MIP compound. In yet another embodiment, the physical spacer is incorporated after the MIP compound is purified. Advantageously, the physical spacer helps to deter swelling and clumping of the fine powder. The physical spacer can be an inert particle. Preferably, the physical spacer is selected from the group consisting of polyethylene oxide, polyvinyl alcohol, bentonite clay, and a combination of the foregoing.

Water is typically added as a solvent in these MIP-forming polymerizations. Hence, the polymers may include hydrogels, which have a significant amount of water in the product. This water can be removed by drying.

Another aspect of the present invention is directed to a molecularly imprinted polymer (also referred to herein interchangeably as MIP or MIP compound) having a complementary structure to, and affinity for, target phosphates. The MIP comprises a plurality of monomers structurally supported by a plurality of cross-linkers, and binding sites for dietary phosphates. Advantageously, the MIP of the present invention are large and non-absorbable from a patient's gastrointestinal (GI) tract.

Monomers useful in the MIP compounds of the present invention preferably possess natural affinities to bind to one or more aspects of a phosphate-containing three-dimensional molecule. These monomers include, without limitation, allyl-amine, methylmethacrylate; hydroxyethylmethacrylate; N,N-di-ethylamino ethyl methacrylate; acrylic acid; alkyl methacrylate; alkylacrylates; arylacrylates; acrylamide; methacrylamide; N-methylacrylamide; N-methylmethacrylamide; styrene; para-hydroxy-styrene; para-amino-styrene; vinylpyridine; para-vinyl benzoic acid; 2-vinyl-2-hydroxypyridine; 3-vinyl-2-hydroxypyridine; 4-vinyl-2-hydroxypyridine; 4-vinylbenzamide; N-alkyl-(4-vinylbenzamide); N,N-dialkyl-(4-vinylbenzamide); N,N′-diethyl(4-vinylphenyl)amidine; acrylonitriles; ethacrylamide; alkacrylamides; alkyl substituted alkyl acrylates in general where the alkyl group is an aliphatic or aromatic group; butadiene; caprolactone; ethylene; propylene; divinylbenzene; ethylene glycol; propylene glycol; dimethylsiloxane; lactide; glycolide; ornithine; vinyl acetate; vinyl alcohol; vinyl chloride; vinyl isobutyl ether; vinyl methyl ether; urethane; isocyanates; isothiocyanates; dimethyl aminoethyl acrylate methyl chloride; [2-(methacryloyloxy)ethyl]trimethylammonium chloride; and vinylpyrrolidone. Preferably, the monomers are acrylates or aromatics. More preferably, the acrylates are selected from the group consisting of methylmethacrylate; hydroxyethylmethacrylate; N,N-di-ethylamino ethyl methacrylate; acrylic acid; and mixtures thereof. More preferably, aromatics are selected from the group consisting of styrene, para-hydroxy-styrene, para-amino-sytrene, vinylpyridine, para-vinyl benzoic acid, and mixtures thereof.

Suitable cross-linkers of the present invention include, without limitation, difunctional acrylates, trifunctional acrylates, tetrafunctional acrylates, difunctional methacrylates, trifunctional methacrylates, tetrafunctional methacrylates, divinyl benzene, alkylene glycol, polyalkylene glycol diacrylates, methacrylates, dialkyldiglycol dicarbonate, dialkyl maleate, dialkyl fumurate, dialkyl itaconate, vinyl esters, ethylene glycol dimethacrylate, ethylene glycol diacrylate, diethylene glycol diacrylate, triethylene glycol diacrylate, tetraethylene glycol diacrylate, vinyl acrylates, vinyl methacrylates, alkyl acrylates, vinyl methacrylates, divinylbenzene, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or bismethacrylamide, including hexamethylene bisacrylamide or hexamethylene bismethacrylamide, di(alkene)tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1,1-dimethyl-2-isocyanaotoethyl acrylate, tetraethylene glycol diacrylate, tetraethylene glycol dimethacrylate, triethylene glycol dimethacrylate, hexanediol dimethacrylate, and hexanediol diacrylate. Preferably, the cross-linker utilized in present invention is di-ethylene glycol diacrylate.

The MIP compounds of the present invention further comprise an inert physical spacer. The spacer is selected for anti-clumping properties. The preferred physical spacer is polyethylene oxide.

In a specific embodiment, the MIP compound of the present invention comprises at least two monomers. In one embodiment, one monomer is polar, and the remaining monomer(s) are less polar.

Unreacted monomers are also removed at this stage. For example, residual monomers that are not part of the cross-linked structure can be washed out.

In another aspect, the invention provides methods for reducing dietary phosphate absorption within a patient by administering an MIP compound of the invention to a patient in need thereof, in an amount sufficient to bind excess dietary phosphates in the patient's gastrointestinal (GI) tract. Preferably, the patient ingests the MIP compound of the invention immediately before eating (e.g., a meal or snack), during eating, or immediately after eating. The methods provide an effective means for decreasing the serum level of phosphates by binding phosphates in the GI tract and excreting them in the patient's feces, thereby preventing its uptake (absorption), without concomitantly increasing the absorption of any clinically undesirable materials, such as calcium or aluminum, as occurs with some other phosphate binders. Thus, the methods of the present invention are useful for treating and/or reducing the severity of symptoms associated with hyperphosphatemia. Elevated serum phosphate (over 5 mg/dL) is commonly present in patients suffering from renal insufficiency, hypoparathyroidism, pseudohypoparathyroidism, acute untreated acromegaly, overmedication with phosphate salts, and acute tissue destruction as occurs during rhabdomyolysis and treatment of malignancies. The dosing regimen will depend, in addition to other factors, upon the affinity of the particular MIP compound and the amount of dietary phosphate present in need of binding.

Patients “in need of treatment” with an MIP compound of the present invention include patients with diseases and/or conditions that can be treated with MIP compounds of the invention to achieve a beneficial therapeutic and/or prophylactic result. A beneficial outcome includes a decrease in the severity of symptoms or delay in the onset of symptoms, increased longevity and/or more rapid or more complete resolution of the disease or condition. For example, a patient in need of treatment typically has elevated serum phosphate levels, hyperphosphatemia, resulting from, for example, impaired kidney function or hypoparathyroidism. Lower serum phosphate levels can be achieved, for example, by inhibiting phosphate uptake in the intestines. A patient “in need of treatment” also includes a patient with chronic renal failure who may have serum phosphate levels within the normal range. Inhibition of phosphate absorption in the intestine or kidneys can slow rate of renal deterioration in these patients, and decrease the risk of cardiovascular events. Other examples of subjects in need of phosphate transport inhibitors include patients with a disease associated with disorders of phosphate metabolism or a disease medicated by impaired phosphate transport function. Examples of diseases and/or disorders of this type include soft tissue calcification, such as cardiovascular calcification, hyperparathyroidism, uremic bone disease, renal bone disease and osteoporosis.

An “effective amount” of an MIP compound disclosed herein is a quantity that results in a beneficial clinical outcome of the condition being treated with the compound compared with the absence of treatment. The amount of MIP compound administered will depend on the degree, severity, and type of the disease or condition, the amount of therapy desired, and the release characteristics of the pharmaceutical formulation. It will also depend on the patient's health, size, weight, age, sex and tolerance to drugs. Typically, the MIP compound is administered for a sufficient period of time to achieve the desired therapeutic effect.

By way of example, the MIP compounds of the present invention can be administered to patients with stages 4 or 5 kidney disease, corresponding to glomerular filtration rates less than approximately 30 ml/min., with the required dose being inversely proportional to the residual renal clearance. Patients with end-stage renal disease (on dialysis) would particularly benefit from the MIP compounds of the present invention.

In certain instances it may be advantageous to co-administer sequentially or simultaneously one or more additional pharmacologically active agents along with an MIP compound of the present invention. In some circumstances, additional phosphate-binding agents (i.e., phosphate sequestrants) can be administered with the MIP compounds of the invention. Examples include pharmaceutically active calcium, aluminum or lanthanum-containing phosphate binders or pharmaceutically active phosphate-binding polymers such as those disclosed in U.S. Pat. Nos. 5,496,545, 5,667,775 and 6,083,495; the contents of which are incorporated herein by reference in their entirety.

Preferably, the method further comprises monitoring the serum phosphate level within the patient before, during, and/or after treatment using the MIP compounds of the present invention. Serum phosphate levels within a patient can be monitored using methods known in the art, such as ion chromatography and spectrophotometric analysis of a blood sample.

The activity of MIP compounds of the present invention can be assessed in vitro using suitable assays, such as the ³³PO₄, Uptake In Rabbit Intestinal Brush Border Membrane Vesicles (BBMV) High Throughput Screening (HTS) assay, and ³³PO₄ uptake in isolated rabbit intestinal rings. MIP compounds of the present invention can also be identified by virtue of their ability to inhibit the absorption of phosphate in vivo, for example, in the gastrointestinal tract of a laboratory animal, as described in Examples 3 and 4.

Decreasing phosphate absorption from the gastrointestinal tract indicates that the percentage of dietary phosphate removed from the gastrointestinal tract by absorption into the body is less when the MIP compounds of the invention are used, than it is when the MIP compounds are not used. This decrease can be determined by comparison of the percentage of dietary phosphate in the feces of an animal while the animal is ingesting the MIP compound with the same percentage when the animal is not ingesting the MIP compound or any other phosphate-complexing agent. Appropriate consideration of changes in phosphate absorption during growth can be accomplished by paired studies of control animals. Further corroborating data for the decrease in gastrointestinal phosphate absorption from an animal may be obtained from comparison of urinary phosphate excretion as a percentage of dietary phosphate before and during an oral trial of the MIP compound that lasts for over a few weeks since the urinary phosphate excretion will decrease when the amount of absorbed phosphate is not sufficient to maintain phosphate homeostasis with the normal urinary phosphate excretion. An additional corroboration of the decrease in gastrointestinal absorption of phosphate can be obtained by measuring serum levels of the species before and during administration of the MIP compound.

The terms “patient”, “recipient”, “subject”, and “host” are used herein interchangeably and, for the purposes of the present invention, include both humans and other animals and organisms, such as experimental animals. Thus, the methods are applicable to both human therapy and veterinary applications, as well as research. Mammalian species which benefit from the methods, MIP compounds, and compositions of the invention include, and are not limited to, apes, chimpanzees, orangutans, humans, monkeys; domesticated animals, including companion animals, such as dogs, cats, guinea pigs, hamsters, Vietnamese pot-bellied pigs, rabbits, and ferrets; laboratory animals, such as rats and mice; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Thus, as used herein, the terms “patient”, “recipient”, “subject”, and “host” are intended to include such human and non-human species.

The MIP compounds of the present invention can be administered to a patient in need thereof or first be incorporated into a pharmaceutical composition. Thus, the present polymers may be systemically administered, e.g., orally, in combination with a pharmaceutically acceptable vehicle such as an inert diluent or an assimilable edible carrier. For example, Remington's Pharmaceutical Sciences (Martin E W [1995] Easton Pa., Mack Publishing Company, 19^{th} ed.) describes formulations which can be used in connection with the present invention. They may be enclosed in hard or soft shell gelatin capsules, may be compressed into tablets, or may be incorporated directly with the food or liquids of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2% to about 60% of the weight of a given unit dosage form. The amount of active compound in such therapeutically useful compositions is such that an effective dosage level will be obtained.

The MIP compounds and pharmaceutical compositions of the invention are preferably non-toxic and stable upon administration. A therapeutically effective amount of a compound is that amount which produces a result or exerts an influence on the particular condition being treated. As used herein, a therapeutically effective amount of an MIP compound means an amount which is effective in decreasing the serum phosphate levels of the patient to which it is administered.

By “non-toxic”, it is meant that when ingested in therapeutically effective amounts, neither the MIP compounds nor any ions released into the body are harmful or are substantially harmful.

By “stable”, it is meant that when ingested in therapeutically effective amounts, no component (e.g., monomer, reagent) of the MIP compound dissolves or otherwise decomposes to form a potentially harmful by-product, and the MIP compound remains substantially intact so that they can transport bound phosphate out of the body.

Preferably, the MIP compound resulting from this imprinting process is in particulate form, and would most ideally be ingested as a capsule, tablet or sprinkled powder at the time of meals. It would be prescribed as a binder for both meals and snacks. The exact dosing regimen would depend on the affinity of the product and the amount of phosphate in the diet in need of binding.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the MIP compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the MIP compound may be incorporated into sustained-release preparations and devices.

Solutions of the MIP compound or its salts can be prepared in water or other suitable solvent, optionally mixed with a nontoxic surfactant. The MIP compounds should be water soluble. However, dispersions can be prepared in glycerol, liquid polyethylene glycols, triacetin, and mixtures thereof and in oils. Under ordinary conditions of storage and use, these preparations contain a preservative to prevent the growth of microorganisms.

The MIP compounds of the present invention are capable of binding phosphate and decrease absorption of dietary phosphate from the gastrointestinal tract, rendering it biologically unavailable. The MIP compounds of the present invention can be described based on the backbone of the its constituent polymer(s), substituents attached to the backbone, functional groups that improve water solubility, and functional groups that permit binding of phosphate.

Water solubility of MIP compounds of the present invention is defined as the ability of the MIP compound to form a homogeneous mixture of an efficacious quantity of the MIP compound with water. Preferably, water solubility of the MIP compounds of the invention imply at least 0.01 gram (g) of the MIP would dissolve in 1000 milliliters (mL) of water, and more preferably, at least 1 g of the polymer would dissolve in 1000 mL of water.

Many water-soluble polymers are known, and higher molecular weight polymers are usually less soluble in water than lower molecular weight polymers of the same composition. (See Thomson, R. A. M., “Methods of polymerization for preparation of water-soluble polymers”, in Chemistry and Technology of Water-Soluble Polymers. Finch, C. A., ed., Plenum, New York, N.Y., 1983; pp 31-70; and Fuchs, O., “Solvents and non-solvents for polymers”, Polymer Handbook, 3.sup.rd Edition, Brandrup, J. and Immergut, E. H., eds. Wiley, New York, N.Y., 1989; pp VII/379-VII/402.)

As previously explained, it is desirable for the MIP compounds of the present invention to be water-soluble. Some polymer backbones contribute to solubility in water. The oxygen atoms in the backbone of some polymers improve water-solubility. Some polymers may benefit from functionalization of side chains to promote water-solubility. Functionalization of the polymer backbone to improve water-solubility may be done by placement of groups which permit hydrogen bonding to water or ionic dissociation in water. Such groups include hydroxyl groups, amine groups, sulfonate groups, phosphonate groups, carbonyl groups, carbamate groups, nitro groups, and carboxylic acid groups. These examples are intended only as exemplifications of functional groups which might improve water-solubility and are not intended to limit the functional groups of this invention. Inclusion of these groups as functional groups of the polymers of the MIP compounds can be done by having the groups already in the monomer when the polymer is prepared or by a separate reaction to introduce the group to a polymer. This technique is well known in the art of polymerization.

The second technique involves introduction onto the polymer of the desired functionality based on transformation of the preexisting functionality of the polymer. Such transformations of functional groups are known in the art of organic chemistry. For example, Comprehensive Organic Transformations: A Guide to Functional Group Preparations, by Richard C. Larock presents many preparative routes for the introduction of various functional groups. This reference includes tables which list the desired functionality, the present functionality, and the reaction sequences which have been reported to accomplish the transformation. Other sources of preparative techniques include Advanced Organic Chemistry: Reactions, Mechanisms, and Structure, Fourth Edition, by Jerry March; Nitration: Methods and Mechanisms by George A. Olah, Ripudaman Malhotra, and Subhash C. Narang; and Advanced Organic Chemistry by Francis A. Carey and Richard J. Sundberg, Plenum Press, NY, 1990.

The liquid pharmaceutical dosage forms can include sterile aqueous solutions or dispersions or sterile powders comprising the MIP compound of the invention which are adapted for the extemporaneous preparation of solutions or dispersions, optionally encapsulated in liposomes. In cases wherein a liquid is prepared, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile solutions are prepared by incorporating the MIP compounds in the required amount in the appropriate solvent with any of the other various ingredients enumerated above, as required, followed by filter sterilization. In the case of sterile powders for the preparation of sterile solutions, the preferred methods of preparation are vacuum drying and the freeze drying techniques, which yield a powder of the active ingredient plus any additional desired ingredient presenting the previously sterile-filtered solutions.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants.

Another embodiment of the MIP compounds produced in accordance with the methods of the present invention is MIP compounds in the form of a hydrogel. Hydrogels are polymer networks that advantageously swell when exposed to water and are hydrophilic, (Peppas et al., “Hydrogels in pharmaceutical formulations,” Eur. J. Pharm. Biopharm. 50, 27-46 (2000). Hydrogels can be formulated to have a low viscosity at room temperature or during administration but also form a hydrogel after ingestion because of the increase in temperature.

Advantageously, MIP compounds of the present invention can swell to roughly ten times their volume upon contact with water. Hydrogel formulations of the present invention can be in forms other than particulates. For example, a liquid or otherwise non-particulate hydrogel formulation may exhibit improved pharmacokinetics when exposed to the conditions of the stomach and GI tract.

As used herein, the terms “phosphate”, “phosphate containing molecule”, and “phosphate containing compound” are referred to interchangeably and intended to include any phosphate bearing chemical entity (e.g., a phosphate containing molecule), including phosphate ion itself, that may be used as a template or target imprint molecule. Preferably, the target imprint molecule is a phosphate that can be found within a patient's diet.

The terms “polar monomer” and “active monomer” are used interchangeably herein to refer to monomers that have an infinity for phosphate and are capable of developing phosphate binding sites, or structurally contributing to phosphate binding sites, when polymerized according the methods of the present invention.

As used herein, the terms “bind”, “bound”, “complex”, “sequester”, and grammatical variations thereof, within the context of a molecularly imprinted polymer and a target phosphate species, are used interchangeably to mean ionic bonding, hydrogen bonding and/or covalent bonding between the molecularly imprinted polymer and its target phosphate species in the GI tract.

As used herein, the term “acrylate” refers to any compound having the formula of H₂C═CHCO₂R, wherein R is an alkyl, aryl, alkaryl or aralkyl group.

The term “alkyl” refers to a group of atoms derived from an alkane by the removal of one hydrogen atom. Thus, the term includes straight or branched chain alkyl moieties or cyclic alkyl moieties including, for example, methyl, ethyl, propyl, isopropyl, butyl, tert-butyl, pentyl, cyclopentyl, isopentyl, neopentyl, hexyl, isohexyl, cyclohexyl, cyclohexylmethyl, 3-methylpentyl, 2,2-methylbutyl, 2,3-dimethylbutyl and the like.

The terms “aryl group” or “aromatic” refers to a group derived from an aromatic hydrocarbon by removal of a hydrogen from the aromatic system. Preferred aryl groups contain phenyl or substituted phenyl groups. Thus, the term “aryl” includes an aromatic carbocyclic radical having a single ring or two condensed rings. This term includes, for example, phenyl or naphthyl.

The terms “comprising”, “consisting of” and “consisting essentially of” are defined according to their standard meaning. The terms may be substituted for one another throughout the instant application in order to attach the specific meaning associated with each term.

As used in this specification and the appended claims, the singular forms “a”, “an”, and “the” include plural reference unless the context clearly dictates otherwise. Thus, for example, a reference to “a monomer” includes more than one such monomer. A reference to “a phosphate” includes more than one such phosphate. A reference to “an MIP” or “an MIP compound” are used herein interchangeably to refer to more than one MIP, and the like.

The term “allyl” refers to a 1-propenyl group of the formula —C₃H₅. The compound allylamine is also called 2-propenylamine.

The term “vinyl” or a “vinyl compound” refers to a vinyl group with the formula CH₂CH—.

The term “difunctional,” “trifunctional,” and “tetrafunctional” refer to compounds having multiple reactive sites. A difunctional compound has two reactive sites, a trifunctional compound has three reactive sites, and a tetrafunctional compound has four reactive sites.

All patents, patent applications, provisional applications, and publications referred to or cited herein, whether supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### EXAMPLE 1

### Synthesis of Molecularly Imprinted Polymer

TABLE 1

| | | Polymer to be made: | | | |
|---|---|---|---|---|---|
| | Compound to be added | 1:1:1 ratio | METAC | HEMA | MMA |
| Crosslinker | di-ethylene glycol diacrylate [mL] | 0.69 | 0.69 | 0.69 | 0.69 |
| Monomer | Hydroxyethyl methacrylate (HEMA) [mL] | 1.01 | 0 | 3.03 | 0 |
| Monomer | Methyl methacrylate (MMA) [mL] | 0.86 | 0 | 0 | 2.58 |
| Monomer | [2-(methacryloyloxy)ethyl] | 2 | 6 | 0 | 0 |
| | trimethylammonium chloride (METAC) [mL] | | | | |
| imprint | KH₂PO₄ [g] for imprint only | 0.4243 | 0.4243 | 0.4243 | 0.4243 |
| molecule | | | | | |
| Diluent | Isopropanol [mL] | 1.2 | 1.2 | 1.2 | 1.2 |
| Initiator | 2,2′-Azobis(2-methylpropionamidine) | 0.0532 | 0.0788 | 0.0448 | 0.0355 |
| | dihydrochloride [g] | | | | |

Four different polymers were synthesized in accordance with the quantities of compounds listed in Table 1. The monomer, diluent, cross-linker and imprint molecule were vortexed and incubated at room temperature for at least three hours to form imprint associations. After three hours, 2,2′-Azobis(2-methylpropionamidine)dihydrochloride (0.0532 g) was added to the monomer mixture, which was then degassed. The mixture was allowed to polymerize while submerged in an oil bath, which was preheated to 50° C. The polymer was finely ground in a food processor and washed and filtered with isopropanol. The polymer was then washed and filtered with deionized water and subsequently washed and filtered with isopropanol. The polymer was dried overnight in a vacuum oven that was preheated to 50° C. The polymer was washed with 40 mL of either HCl or deionized water and allowed to sit overnight in an incubator at 37° C. The polymer solution was centrifuged, and the supernatant removed and tested for phosphate concentration. The elution was repeated until the phosphate concentration in the supernatant is negligible. 20 mL of water was added to the polymer, and the pH was adjusted to 7 using either HCl or NaOH. The polymer was filtered out and dried overnight in a vacuum oven that was preheated to 50° C.

The 1:1:1 polymer is an example of a MIP compound possessing a polar, active monomer ([2-(methacryloyloxy)ethyl]trimethylammonium chloride; METAC) and two less polar, relatively inactive monomers (HEMA and MMA).

Phosphate binding using just METAC was limited by tight cross-linking and inadequate exposure of the active sites. To optimize macroporosity and fluid flow, the 1:1:1 MIP compound was synthesized.

### EXAMPLE 2

### Phosphate Uptake of Molecularly Imprinted Polymers

2.72 g of KH₂PO₄, 4.676 g of NaCl, and 3.18 g of Na₂CO₃ were added to a 1 L Erlenmeyer flask. Enough deionized water was added to fill the flask up to 900 mL and was agitated to partially dissolve the powders. The pH was adjusted to 7.0 with 1M HCl or diluted NaOH. Additional deionized water was added to the mixture to make a 1 L solution. The solution was mixed for at least 15 minutes until all of the solids dissolved.

The phosphate solution was warmed in a 37° C. water bath. 100 mg samples of the following polymers were weighed out: a MIP from Sample 1, which is composed from a 1:1:1 ratio of the less polar (relatively inactive) monomers hydroxyethyl methacrylate (HEMA) and methyl methacrylate (MMA) and the polar (active) monomer [2-(methacryloyloxy)ethyl]trimethylammonium chloride (METAC) with a polyethylene oxide (PEO) spacer; a compound having a 1:1:1 ratio of HEMA, MMA, and METAC without molecular imprinting and the PEO spacer; and two sevelamer hydrochloride samples dissolved in water. 30 mL of the phosphate solution was added to each sample, respectively. The samples in solution were vortexed for about 30 seconds and placed in a 37° C. incubator with the tubes rotating for three hours. The incubated samples were centrifuged for about 5 minutes and the supernatant filtered.

The filtrates were analyzed for phosphate binding in an Inductively Coupled Plasma Spectrometer (ICP), and the results are shown in Table 2. Phosphate binding may be evaluated using any means known in the art, however, such as ionic chromatography.

With acid elution of the templates, the 1:1:1 MIP compound of Example 1 bound 0.097 meq/g compared to 0.022 meq/g nonimprinted polymer and 0.17 meq/g sevelamer. Therapuetic MIPs were thus synthesized that effectively bound phosphate. The exemplified method may be optimized to increase phosphate binding by varying parameters, such as, the monomers utilized (e.g., proportion of polar monomers to less polar monomers), cross-linking agents used, and choice of extraction procedures.

TABLE 2

| | 1^{st} | 2^{nd} | | | |
|---|---|---|---|---|---|
| | reading | reading | | | |
| | mean | mean | Average of | Concen- | |
| | concen- | concen- | 1^{st} and 2^{nd} | tration | Amount of |
| | tration | tration | reading | Change | Phosphate |
| | [mg/l] = | [mg/l] = | [mg/l] = | [mg/l] = | Bound |
| | ppm | ppm | ppm | ppm | [meq/g] |
| 20 mM P | 615 | 619 | 617 | 0 | 0 |
| (stock | | | | | |
| phosphate | | | | | |
| solution) | | | | | |
| 1:1:1 with P | 585 | 587 | 586 | 31 | 0.097 |
| and PEO | | | | | |
| 1:1:1 | 606 | 614 | 610 | 7 | 0.022 |
| without P | | | | | |
| and PEO | | | | | |
| Sevelamer | 565 | 559 | 562 | 55 | 0.172 |
| water | | | | | |
| dissolved | | | | | |
| sample 1 | | | | | |
| Sevelamer | 559 | 556 | 557.5 | 59.5 | 0.186 |
| water | | | | | |
| dissolved | | | | | |
| sample 2 | | | | | |

### SUMMARY OF RESULTS

To achieve high-affinity low-cost oral phosphate binding, the present inventors developed methodology for synthesizing targeted MIPs: monomers self-assemble around a template, which is then eluted, yielding active polymer in a lock-key schema. Cationic monomers were identified, and one ([2-(methacryloyloxy)ethyl]trimethylammonium chloride, METAC) was utilized for syntheses. In a KH₂PO₄ phosphate solution, it was polymerized using a water soluble azo bis initiator and di-ethylene glycol di-acrylate as the cross-linker. The resultant polymer block was dried, ground, phosphate eluted, redried and ground to a powder. To optimize particle dispersion in water, polyetheylene oxide was added (10% weight). Phosphate uptake by the MIPs was evaluated by atomic absorption using a NaCl and carbonate solution of 20 mM KH₂PO₄ at pH 7; binding was compared to that of nonimprinted polymer and sevelamer HCl. Imprinting at two pHs (4.3 and 7), and elutions in either base or acid were evaluated. Scanning EM of the MIPs were obtained.

Phosphate binding using just METAC was felt to be limited by tight cross-linking and inadequate exposure of the active sites. To optimize macroporosity and fluid flow, two approaches were used to open up the structure. First, co-polymerization was carried out with other monomer spacers (methyl methacrylate and hydroxyethyl methacrylate) in 1:1 or 1:1:1 ratios. With acid elution of the template, the latter MIP bound 0.097 meq/g compared to 0.022 meq/g nonimprinted polymer, and 0.17 meq/g sevelamer, as shown in Table 2. One can also synthesize MIPs around relatively hydrophobic diluents in the aqueous templating solution (i.e., octanol or isopropanol) which are then removed by heating. The degree of cross-linking also affected hydrogel formation when the final MIPs were exposed to aqueous test solutions, swelling up to nearly 10 times volume.

Therapeutic MIPs were thus synthesized, proving effective phosphate binding and imprinting, thereby demonstrating feasibility of this new low cost novel technology. As indicated above, the methodology may be optimized for phosphate binding by testing alternate monomers and spacers to maximize exposure of the high-affinity sites, for example. The potential pharmacokinetic benefits of hydrogel formulation may also be explored.

### EXAMPLE 3

### Pharmaceutical Composition for Oral Administration

10 parts by weight of polyethylene oxide is added to ten parts by weight of the polymer produced in Example 1. The resulting mixture is combined with deionized water to form a fine paste. The paste is allowed to dry overnight in a vacuum oven, which is preheated to 50° C.

### EXAMPLE 4

### Rat Dietary Phosphorus Excretion Model

Six 6-8 week old Sprague-Dawley rats are placed in metabolic cages and fed semi-purified rodent chow powder containing 0.28% inorganic phosphorus. The diets are supplemented with an MIP compound of the present invention or micro-crystalline cellulose; the animals serve as their own controls by receiving cellulose or the MIP compound in randomized order. The rats are fed ad libitum for three days to acclimate to the diet. Feces excreted during the next 48 hours are collected, lyophilized, and ground into powder. The inorganic phosphate content is determined according to the method of Taussky and Shorr: Microdetermination of Inorganic Phosphate. One gram of powdered feces is burned to remove carbon, then ashed in a 600° C. oven. Concentrated HCl is then added to dissolve the phosphorus. The phosphorus is determined with ferrous sulfate-ammonium molybdate reagent. Intensity of the blue color is determined at 700 nm on a Perkin-Elmer spectrophotometer through a 1 cm cell.

### EXAMPLE 5

### Urinary Phosphate Excretion in Partially Nephrectomized Rats

Sprague-Dawley rats, approximately 8 weeks old, are 75% nephrectomized. One kidney is surgically removed; approximately 50% of the renal artery flow to the contralateral kidney is ligated. The animals are fed a semi-purified rodent chow containing 0.385% inorganic phosphorus and either an MIP compound of the present invention or cellulose. Urine is collected and analyzed for phosphate content on specific days. Absorbed dietary phosphate is excreted into the urine to maintain serum phosphate. If the animals receiving the MIP compound demonstrate a trend toward reduced phosphate excretion, it is indicative of reduced phosphate absorption.

1. A molecularly imprinted polymer (MIP) compound comprising:
a) a polymer, electrostatically imprinted with a phosphate, comprising at least one monomer exhibiting an affinity for phosphate ions, phosphate containing molecules, or a combination of both, at least one cross-linking agent for structurally supporting the at least one monomer, and a plurality of binding sites for binding to phosphate ions, phosphate containing molecules, or a combination of both, wherein the binding sites are accessible by pores; or
b) a polymer, electrostatically imprinted with a phosphate, comprising at least one monomer exhibiting an affinity for phosphate ions, phosphate containing molecules, or a combination of both, at least one cross-linking agent for structurally supporting the at least one monomer, and a plurality of binding sites for binding to phosphate ions, phosphate containing molecules, or a combination of both, wherein the binding sites are accessible by pores, and a physical spacer.

2. The MIP compound according to claim 1, wherein the at least one monomer is selected from the group consisting of allylamine, methylmethacrylate; hydroxyethylmethacrylate; N,N-di-ethylamino ethyl methacrylate; acrylic acid; alkyl methacrylate; alkylacrylate; arylacrylate; acrylamide; methacrylamide; N-methylacrylamide; N-methylmethacrylamide; styrene; para-hydroxy-styrene; para-amino-styrene; vinylpyridine; para-vinyl benzoic acid; 2-vinyl-2-hydroxypyridine; 3-vinyl-2-hydroxypyridine; 4-vinyl-2-hydroxypyridine; 4-vinylbenzamide; N-alkyl-(4-vinylbenzamide); N,N-dialkyl-(4-vinylbenzamide); N,N′-diethyl(4-vinylphenyl)amidine; acrylonitriles; ethacrylamide; alkacrylamides; alkyl substituted alkyl acrylates; butadiene; caprolactone; ethylene; propylene; divinylbenzene; ethylene glycol; propylene glycol; dimethylsiloxane; lactide; glycolide; ornithine; vinyl acetate; vinyl alcohol; vinyl chloride; vinyl isobutyl ether; vinyl methyl ether; urethane; isocyanates; isothiocyanates; dimethyl aminoethyl acrylate methyl chloride; [2-(methacryloyloxy)ethyl]trimethylammonium chloride; vinylpyrrolidone; and combinations of any of the foregoing.

3. The MIP compound according to claim 1, wherein the at least one cross-linking agent is selected from the group consisting of difunctional acrylate; trifunctional acrylate; tetrafunctional acrylate; difunctional methacrylate; trifuncitonal methacrylate; tetrafunctional methacrylate; divinyl benzene; alkylene glycol; polyalkylene glycol diacrylate; methacrylate; dialkyldiglycol dicarbonate; dialkyl maleate; dialkyl fumurate; dialkyl itaconate; vinyl ester; ethylene glycol dimethacrylate; ethylene glycol diacrylate; di-ethylene glycol diacrylate; triethylene glycol diacrylate; tetraethylene glycol diacrylate; vinyl acrylate; vinyl methacrylate; alkyl acrylate; divinylbenzene; diallyldiglycol dicarbonate; diallyl maleate; diallyl fumarate; diallyl itaconate; vinyl ester; divinyl oxalate; divinyl malonate; diallyl succinate; triallyl isocyanurate; dimethacrylate of bisphenol A; diacrylate of bisphenol A; dimethacrylate of ethoxylated bisphenol A; diacrylate of ethoxylated bispherol A; methylene; polymethylene bisacrylamide; bismethacrylamide; hexamethylene bisacrylamide; hexamethylene bismethacrylamide; di(alkene)tertiary amine; trimethylol propane triacrylate; pentaerythritol tetraacrylate; divinyl ether; divinyl sulfone; diallyl phthalate; triallyl melamine; 2-isocyanatoethyl methacrylate; 2-isocyanatoethylacrylate; 3-isocyanatopropylacrylate; 1-methyl-2-isocyanatoethyl methacrylate; 1,1-dimethyl-2-isocyanaotoethyl acrylate; tetraethylene glycol diacrylate; tetraethylene glycol dimethacrylate; triethylene glycol dimethacrylate; hexanediol dimethacrylate; hexanediol diacrylate; and a combination of any of the foregoing.

4. The MIP compound according to claim 1, comprising about 3 wt % to about 35 wt % of the at least one cross-linker.

5. The MIP compound according to claim 1, wherein the physical spacer comprises polyethylene oxide, polyvinyl alcohol, bentonite clay, or a combination of the foregoing.

6. The MIP compound according to claim 1, wherein the compound is formulated as a particulate or a hydrogel.

7. A method for treating a patient having an excess of phosphate ions and/or phosphate containing molecules in the gastrointestinal tract, the method comprising:
a) administering an effective amount of a molecularly imprinted polymer (MIP) compound according to claim 1 to the gastrointestinal tract of the patient;
b) administering an effective amount of a MIP compound according to claim 1 to the gastrointestinal tract of the patient and monitoring serum phosphate levels in the patient;
c) administering an effective amount of a MIP compound according to claim 1 to the gastrointestinal tract of the patient, monitoring serum phosphate levels in the patient, and co-administering, sequentially or simultaneously, at least one pharmacologically active agent; or
d) administering an effective amount of a MIP compound according to claim 1 to the gastrointestinal tract of the patient and co-administering, sequentially or simultaneously, at least one pharmacologically active agent;
wherein the at least one monomer of the MIP compound is selected from the group consisting of:
i) a monomer exhibiting an affinity for phosphate ions, phosphate containing molecules, or a combination of both;
ii) a monomer exhibiting an affinity for phosphate ions, phosphate containing molecules, or a combination of both selected from the group consisting of allylamine; methylmethacrylate; hydroxyethylmethacrylate; N,N-di-ethylamino ethyl methacrylate; acrylic acid; alkyl methacrylate; alkylacrylate; arylacrylate; acrylamide; methacrylamide; N-methylacrylamide; N-methylmethacrylamide; styrene; para-hydroxy-styrene; para-amino-styrene; vinylpyridine; para-vinyl benzoic acid; 2-vinyl-2-hydroxypyridine; 3-vinyl-2-hydroxypyridine; 4-vinyl-2-hydroxypyridine; 4-vinylbenzamide; N-alkyl-(4-vinylbenzamide); N,N-dialkyl-(4-vinylbenzamide); N,N′-diethyl(4-vinylphenyl)amidine; acrylonitrile; ethacrylamide; alkacrylamide; alkyl substituted alkyl acrylate; butadiene; caprolactone; ethylene; propylene; divinylbenzene; ethylene glycol; propylene glycol; dimethylsiloxane; lactide; glycolide; ornithine; vinyl acetate; vinyl alcohol; vinyl chloride; vinyl isobutyl ether; vinyl methyl ether; urethane; isocyanates; isothiocyanates; dimethyl aminoethyl acrylate methyl chloride; [2-(methacryloyloxy)ethyl]trimethylammonium chloride; vinylpyrrolidone; and combinations of any of the foregoing;
iii) a monomer exhibiting an affinity for phosphate ions, phosphate containing molecules, or a combination of both selected from the group consisting of hydroxyethyl methacrylate, methyl methacrylate, [2-(methacryloyloxy)ethyl]trimethylammonium chloride, acrylic acid, allylamine, and combinations of any of the foregoing; and
iv) a monomer exhibiting an affinity for phosphate ions, phosphate containing molecules, or a combination of both selected from the group consisting of styrene, parahydroxy-styrene, para-amino styrene, vinyl-pyridine, para-vinyl benzoic acid, and combinations of the foregoing;
wherein the at least one cross-linking agent of the MIP compound is selected from the group consisting of:
v) difunctional acrylate; trifunctional acrylate; tetrafunctional acrylate; difunctional methacrylate; trifunctional methacrylate; tetrafunctional methacrylate; divinyl benzene; alkylene glycol; polyalkylene glycol diacrylate; methacrylate; dialkyldiglycol dicarbonate; dialkyl maleate; dialkyl fumurate; dialkyl itaconate; vinyl ester; ethylene glycol dimethacrylate; ethylene glycol diacrylate; diethylene glycol diacrylate; triethylene glycol diacrylate; tetraethylene glycol diacrylate; vinyl acrylate; vinyl methacrylate; alkyl acrylate; divinylbenzene; diallyldiglycol dicarbonate; diallyl maleate; diallyl fumarate; diallyl itaconate; vinyl ester; divinyl oxalate; divinyl malonate; diallyl succinate; triallyl isocyanurate; dimethacrylate of bisphenol A; diacrylate of bisphenol A; dimethacrylate of ethoxylated bisphenol A; diacrylate of ethoxylated bispherol A; methylene; polymethylene bisacrylamide; bismethacrylamide; hexamethylene bisacrylamide; hexamethylene bismethacrylamide; di(alkene)tertiary amine; trimethylol propane triacrylate; pentaerythritol tetraacrylate; divinyl ether; divinyl sulfone; diallyl phthalate; triallyl melamine; 2-isocyanatoethyl methacrylate; 2-isocyanatoethylacrylate; 3-isocyanatopropylacrylate; 1-methyl-2-isocyanatoethyl methacrylate; 1,1-dimethyl-2-isocyanaotoethyl acrylate; tetraethylene glycol diacrylate; tetraethylene glycol dimethacrylate; triethylene glycol dimethacrylate; hexanediol dimethacrylate; hexanediol diacrylate; or a combination of any of the foregoing; and
vi) di-ethylene glycol diacrylate; and
wherein the physical spacer of the MIP compound is selected from the group consisting of:
vii) an inert particle; and
viii) an inert particle selected from the group consisting of polyethylene oxide, polyvinyl alcohol, bentonite clay, and a combination of any of the foregoing.

8. The method according to claim 7, wherein each of the administering steps comprise the patient ingesting the molecularly imprinted polymers.

9. The method according to claim 7, wherein the pharmacologically active agent is a phosphate sequestrant.

10. The method according to claim 7, wherein the MIP compound comprises two polar monomers, wherein the degree of polarity varies to the extent that the less polar monomer is relatively inactive when compared to the more polar monomer.

11. The method according to claim 7, wherein the weight percentage of cross-linking agent in the molecularly imprinted polymer is within the range of about 3 wt % to about 35 wt %.

12. The method according to claim 7, comprising administering the MIP compound as a hydrogel, a particulate, or a particulate formulated as a capsule or tablet.

13. A method for synthesizing a molecularly imprinted polymer (MIP) compound 1 having a complementary structure to phosphate ions, wherein the method comprises:
a) providing a mixture comprising: at least one monomer; at least one diluent; and at least one cross-linking agent;
b) contacting a target imprint molecule with the provided mixture so that the at least one monomer self-assembles around the target imprint molecule in a lowest energy state for the at least one monomer;
c) polymerizing the at least one monomer, wherein the polymerized monomers are supported by the at least one cross-linking agent, wherein the polymerized monomers are electrostatically imprinted to the target imprint molecule, and
d) removing the target imprint molecule and the at least one diluent from the polymerized monomers, whereby a MIP compound according to claim 1 is formed; or
e) providing a mixture comprising: at least one monomer; at least one pore forming diluent; and at least one cross-linking agent;
f) contacting a target imprint molecule with the provided mixture so that the at least one monomer self-assembles around the target imprint molecule in a lowest energy state for the at least one monomer;
g) polymerizing the at least one monomer, wherein the polymerized monomers are supported by the at least one cross-linking agent, wherein the polymerized monomers are electrostatically imprinted to the target imprint molecule;
h) removing the target imprint molecule and the at least one diluent from the polymerized polymers, whereby a MIP compound according to claim 1 is formed; and
i) incorporating a physical spacer throughout the MIP compound or the polymerized monomers;
wherein the at least one monomer is selected from the group consisting of:
i) a monomer exhibiting an affinity for phosphate ions, phosphate containing molecules, or a combination of both;
ii) a monomer exhibiting an affinity for phosphate ions, phosphate containing molecules, or a combination of both selected from the group consisting of allylamine; methylmethacrylate; hydroxyethylmethacrylate; N,N-di-ethylamino ethyl methacrylate; acrylic acid; alkyl methacrylate; alkylacrylate; arylacrylate; acrylamide; methacrylamide; N-methylacrylamide; N-methylmethacrylamide; styrene; para-hydroxy-styrene; para-amino-styrene; vinylpyridine; para-vinyl benzoic acid; 2-vinyl-2-hydroxypyridine; 3-vinyl-2-hydroxypyridine; 4-vinyl-2-hydroxypyridine; 4-vinylbenzamide; N-alkyl-(4-vinylbenzamide); N,N-dialkyl-(4-vinylbenzamide); N,N′-diethyl(4-vinylphenyl)amidine; acrylonitrile; ethacrylamide; alkacrylamide; alkyl substituted alkyl acrylate; butadiene; caprolactone; ethylene; propylene; divinylbenzene; ethylene glycol; propylene glycol; dimethylsiloxane; lactide; glycolide; ornithine; vinyl acetate; vinyl alcohol; vinyl chloride; vinyl isobutyl ether; vinyl methyl ether; urethane; isocyanates; isothiocyanates; dimethyl aminoethyl acrylate methyl chloride; [2-(methacryloyloxy)ethyl]trimethylammonium chloride; vinylpyrrolidone; and combinations of any of the foregoing;
iii) a monomer exhibiting an affinity for phosphate ions, phosphate containing molecules, or a combination of both selected from the group consisting of hydroxyethyl methacrylate, methyl methacrylate, [2-(methacryloyloxy)ethyl]trimethylammonium chloride, acrylic acid, allylamine and combinations of any of the foregoing; and
iv) a monomer exhibiting an affinity for phosphate ions, phosphate containing molecules, or a combination of both selected from the group consisting of styrene, parahydroxy-styrene, para-amino styrene, vinyl-pyridine, para-vinyl benzoic acid, and combinations of the foregoing;
wherein the at least one cross-linking agent is selected from the group consisting of:
v) difunctional acrylate; trifunctional acrylate; tetrafunctional acrylate; difunctional methacrylate; trifunctional methacrylate; tetrafunctional methacrylate; divinyl benzene; alkylene glycol; polyalkylene glycol diacrylate; methacrylate; dialkyldiglycol dicarbonate; dialkyl maleate; dialkyl fumurate; dialkyl itaconate; vinyl ester; ethylene glycol dimethacrylate; ethylene glycol diacrylate; diethylene glycol diacrylate; triethylene glycol diacrylate; tetraethylene glycol diacrylate; vinyl acrylate; vinyl methacrylate; alkyl acrylate; divinylbenzene; diallyldiglycol dicarbonate; diallyl maleate; diallyl fumarate; diallyl itaconate; vinyl ester; divinyl oxalate; divinyl malonate; diallyl succinate; triallyl isocyanurate; dimethacrylate of bisphenol A; diacrylate of bisphenol A; dimethacrylate of ethoxylated bisphenol A; diacrylate of ethoxylated bispherol A; methylene; polymethylene bisacrylamide; bismethacrylamide; hexamethylene bisacrylamide; hexamethylene bismethacrylamide; di(alkene)tertiary amine; trimethylol propane triacrylate; pentaerythritol tetraacrylate; divinyl ether; divinyl sulfone; diallyl phthalate; triallyl melamine; 2-isocyanatoethyl methacrylate; 2-isocyanatoethylacrylate; 3-isocyanatopropylacrylate; 1-methyl-2-isocyanatoethyl methacrylate; 1,1-dimethyl-2-isocyanaotoethyl acrylate; tetraethylene glycol diacrylate; tetraethylene glycol dimethacrylate; triethylene glycol dimethacrylate; hexanediol dimethacrylate; hexanediol diacrylate; or a combination of any of the foregoing; and
vi) di-ethylene glycol diacrylate;
wherein the physical spacer is selected from the group consisting of:
vii) an inert particle; and
viii) an inert particle selected from the group consisting of polyethylene oxide, polyvinyl alcohol, bentonite clay, and a combination of any of the foregoing; and
wherein the at least one diluent comprises one diluent comprising isopropanol; the at least one diluent comprises one diluent comprising hexane; or the at least one dilent comprises two or more diluents selected from the group consisting of methanol, ethanol, t-butanol, octanol, isopropanol, hexane, and combinations of any of the foregoing.

14. The method according to claim 13, wherein the provided mixture comprises about 3 parts by volume of the at least one monomer per about 9 parts by volume of the at least one cross-linking agent.

15. The method according to claim 13, wherein the provided mixture comprises one part by weight of the at least one diluent per one part by weight of the at least one monomer.

16. The method according to claim 13, wherein the at least one monomer comprises a first polar monomer and a second polar monomer, and wherein the polarity of the first monomer is relatively inactive compared to the polarity of the second polar monomer.

17. The method according to claim 16, wherein the first polar monomer is selected from the group consisting of hydroxyethyl methacrylate, methyl methacrylate, and a combination of both; and wherein the second polar monomer is selected from the group consisting of [2-(methacryloyloxy)ethyl]trimethyl ammonium, allylamine, and a combination of both.

18. The method according to claim 16, wherein the at least one monomer comprises about one part by weight of the first polar monomer and about two parts by weight of the second polar monomer.

19. The method according to claim 13, wherein the provided mixture comprises one part by weight of the at least one diluent and one part by weight of the at least one monomer.

20. The method according to claim 13, wherein the provided mixture comprises two diluents; and wherein the provided mixture further comprises one part by weight of each of the two diluents and one part by weight of the at least one monomer.

21. The method according to claim 13, comprising the MIP compound comprising about 3 wt % to about 35 wt % of the at least one cross-linking agent.

22. The method according to claim 13, wherein the contacting step takes place at a temperature within the range of about 17° C. to about 77° C.

23. The method according to claim 13, wherein the polymerizing step is initiated using an initiator selected from the group consisting of benzol peroxide; acetyl peroxide; lauryl peroxide; azobisisobutyronitrile; 2,2′-Azobis(2-methylpropionamidine)dihydrochloride; t-butyl peracetate; cumyl peroxide; t-butyl peroxide; t-butyl hydroperoxide; bis(isopropyl)peroxy-dicarbonate; benzoin methyl ether; 2,2′-azobis(2,4-dimethylvaleronitrile); tert-butyl peroctoate; phtalic peroxide; diethoxyacetophenon; tert-butyl peroxyypivalate; diethoxyacetophenone; 1-hydroxycyclohexyl phenyl ketone; 2,2-dimethoxy-2-phenyl-acetophenone; phenothiazine; diisopropylxanthogen disulfide; and a combination of any of the foregoing.

24. The method according to claim 13, wherein the removing step comprises:
a) a plurality of washings and filtrations, and wherein the washings comprise alternating alcohol and aqueous washings;
b) grinding the MIP compound;
c) comprises drying the MIP compound, and wherein the drying step comprises freeze drying or vacuum drying; or
d) a combination of the foregoing.
